(19) United States
(12) Patent Application Publication
(10) Pub. No.: US 2019/0392944 A1
(43) Pub. Date: Dec. 26, 2019

(54) METHOD AND WORKSTATIONS FOR A DIAGNOSTIC SUPPORT SYSTEM

(71) Applicant: General Electric Company, Schenectady, NY (US)

(72) Inventors: Eigil Samset, Oppegard (NO); Mark Kohls, Delafield, WI (US)

(21) Appl. No.: 16/016,291

(22) Filed: Jun. 22, 2018

(51) Int. Cl. G16H 30/40; G06F 17/30; G16H 50/20; G06T 7/00

(52) U.S. Cl. CPC G16H 30/40; G06F 17/30274; G06F 3/0482; G06T 7/0012; G16H 50/20

(57) ABSTRACT

Methods and systems are provided for automating analysis of diagnostic medical images. In one example, a method includes obtaining a set of medical images of a patient, automatically generating a set of clinical parameters from the set of medical images, automatically identifying a clinical finding of the patient based on at least one selected clinical parameter of the set of clinical parameters, outputting a graphical user interface for display on a display device, the graphical user interface including a visualization of the clinical finding and a link within the visualization of the clinical finding, and responsive to selection of the link, outputting, for display within the graphical user interface, a visualization of the at least one selected clinical parameter.

### FIELD

Embodiments of the subject matter disclosed herein relate to medical imaging, and more particularly, to automating medical imaging diagnostic workflows.

### BACKGROUND

Medical imaging diagnostic workflows typically include acquisition of a plurality of medical images of a patient, which are then analyzed by a clinician, such as a radiographer or technician, to measure various aspects of anatomical features present within the medical images. The medical images and measurements may then be analyzed by another clinician, such as a cardiologist or radiologist, to determine if any abnormal clinical findings are present.

### BRIEF DESCRIPTION

In one embodiment, a method comprises obtaining a set of medical images of a patient, automatically generating a set of clinical parameters from the set of medical images, automatically identifying a clinical finding of the patient based on at least one selected clinical parameter of the set of clinical parameters, outputting a graphical user interface for display on a display device, the graphical user interface including a visualization of the clinical finding and a link within the visualization of the clinical finding, and responsive to selection of the link, outputting, for display within the graphical user interface, a visualization of the at least one selected clinical parameter.

In this way, a reviewing clinician, such as a cardiologist, may be presented with a set of automatically-determined clinical findings. The clinician may then drill down from the clinical findings to the measurements and ultimately the medical images from which the clinical findings were based, in order to confirm the accuracy and/or the correctness of the clinical findings and submit a final report. By starting with a set of automatically-generated clinical findings and working backwards to the medical images, the process of reviewing a diagnostic imaging exam may be expedited and aspects of the exam prone to error or inconsistencies (e.g., human-to-human variability in taking measurements of features of the images) may be performed in a uniform manner.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example imaging system.

FIG. 2 shows a flow chart illustrating an example method for automatically determining a set of clinical findings during a diagnostic exam.

FIGS. 3A and 3B show a flow chart illustrating an example method for displaying the results of the automatically determined set of clinical findings.

FIG. 4 shows an example graphical user interface for displaying a set of diagnostic exams.

FIGS. 5-6 show an example graphical user interface for displaying clinical findings of a patient diagnostic exam.

### DETAILED DESCRIPTION

Structural and/or functional abnormalities of certain anatomical features of a patient may be diagnosed using medical imaging, such as ultrasound imaging or magnetic resonance imaging (MRI). For example, heart abnormalities may be diagnosed using ultrasound images in a process commonly referred to as an echocardiogram. During a standard echocardiogram, a patient is examined with a full echocardiogram protocol by a sonographer on an ultrasound imaging scanner. The images acquired during the exam are transferred to a central storage device, such as a hospital picture archiving and communication system (PACS). The images are analyzed by a sonographer and a set of measurements (typically 50-100 records) are made from the images. The images and measurements are reviewed by a cardiologist, and a set of clinical findings/conclusions are made and submitted as a report for the patient's medical record.

This standard workflow is time consuming (as measuring the images may take 30% of the total time for conducting an echocardiogram), prone to user variability (as ultrasound measurements suffer from low reproducibility), and may be prone to misdiagnosis. Also, many patients are sent to expensive imaging (e.g., MM or CT) because of an inconclusive echocardiogram examination. The misdiagnosis and referral for other imaging modalities may be at least in part due to human error in taking measurements and/or person-to-person variability in how measurements are taken and interpreted.

Thus, according to embodiments disclosed herein, the above drawbacks of a standard diagnostic imaging workflow may be avoided by a tool that enforces the same rigor and detailed attention to every exam. A diagnostic imaging workflow according to the embodiments described herein may include a reversal of the above-described standard workflow, enabled through Artificial Intelligence (AI). In the workflow according to the disclosure, the patient is examined with a full imaging protocol by a clinician on a suitable medical imaging modality. For example, a full echocardiogram protocol may be executed by a sonographer on an ultrasound scanner. The resultant patient medical images are transferred to an AI enabled device (e.g., in the cloud). All the images are automatically analyzed using a combination of AI (e.g., to automate measurements and identify pathology in the images) and evidence-based medicine (e.g., published guidelines, normal-ranges, study results) to produce a list of findings/conclusions. A clinician, typically a cardiologist or radiologist, over-reads the automated findings, and can drill down into the reasoning made by the algorithm for each AI-based finding.

A user interface may be presented to the clinician, allowing the clinician to get an overview of the severity and number of findings for each patient examined on a specific day or week. Furthermore, for each patient, the clinician will be presented a pre-populated list of clinical findings. By clicking at a clinical finding, the clinician may be shown, via the graphical user interface, how the AI-enabled device identified that particular clinical finding. This may include a measurement that is above/below normal range, or a clinical finding observed by the AI in an image. By choosing a measurement, the clinician may be shown which image or set of images was used to make the automated measurement and the clinician may visualize the measurement (e.g. via endpoints). The clinician may then approve the clinical finding for inclusion in a final report, or omit the clinical finding from the final report if the clinician disagrees with the clinical finding or does not think sufficient evidence is available to conclude the clinical finding is present (e.g., the image quality is too low or not enough measurements were available).

The automated measurement of relevant clinical parameters, subsequent identification of clinical findings, and presentation of the findings to a clinician are performed by utilizing a graphical user interface (GUI) with a data model linking conclusions, measurements, evidence (e.g. guidelines), and images, as well as a Deep Neural Network or other type of model that can make automatic measurements in images (e.g., ejection fraction, dimensions, Doppler measurements).

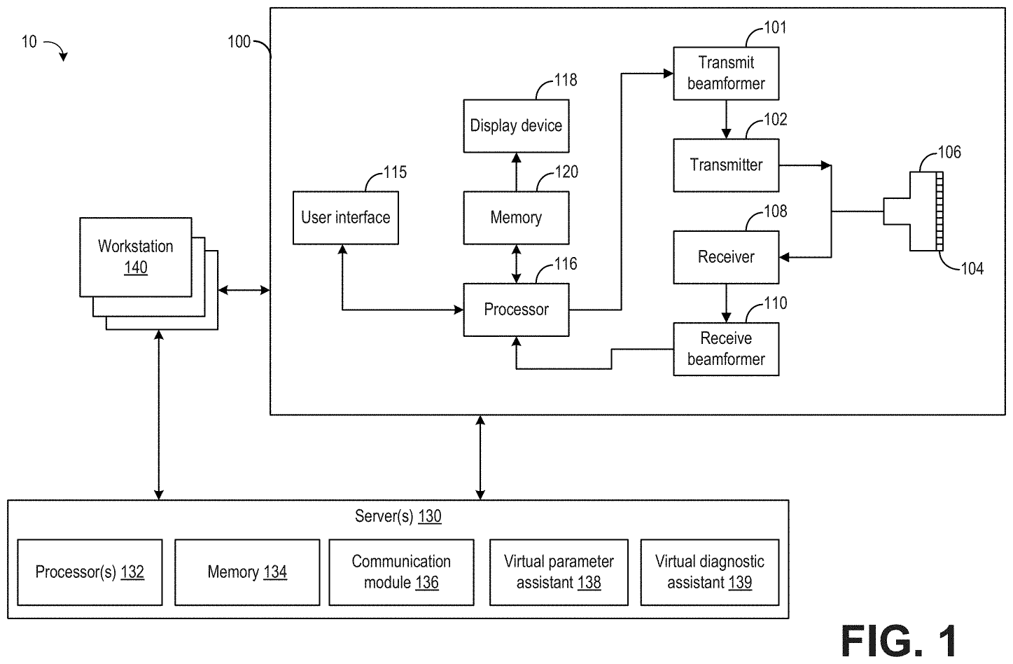

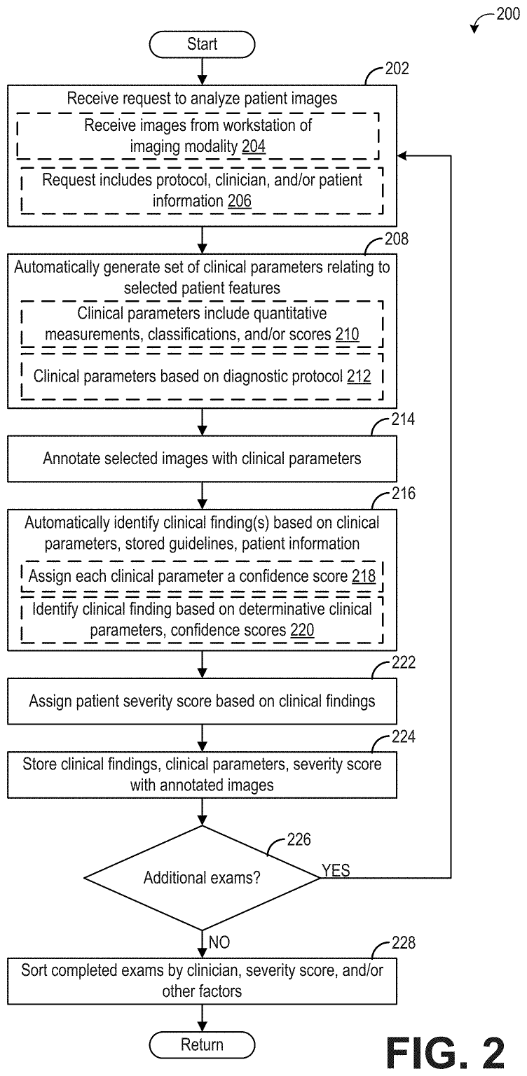

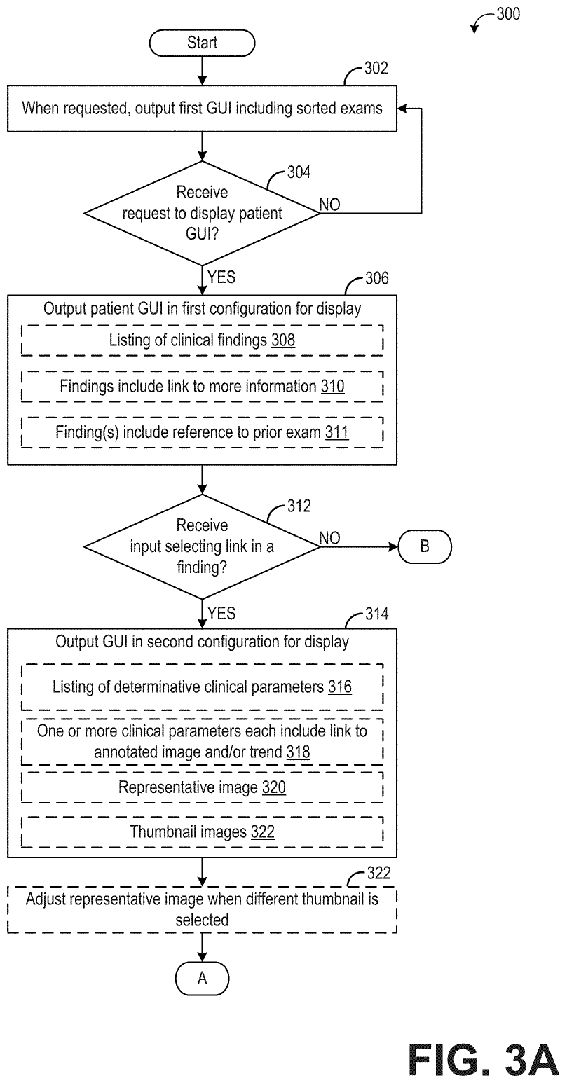

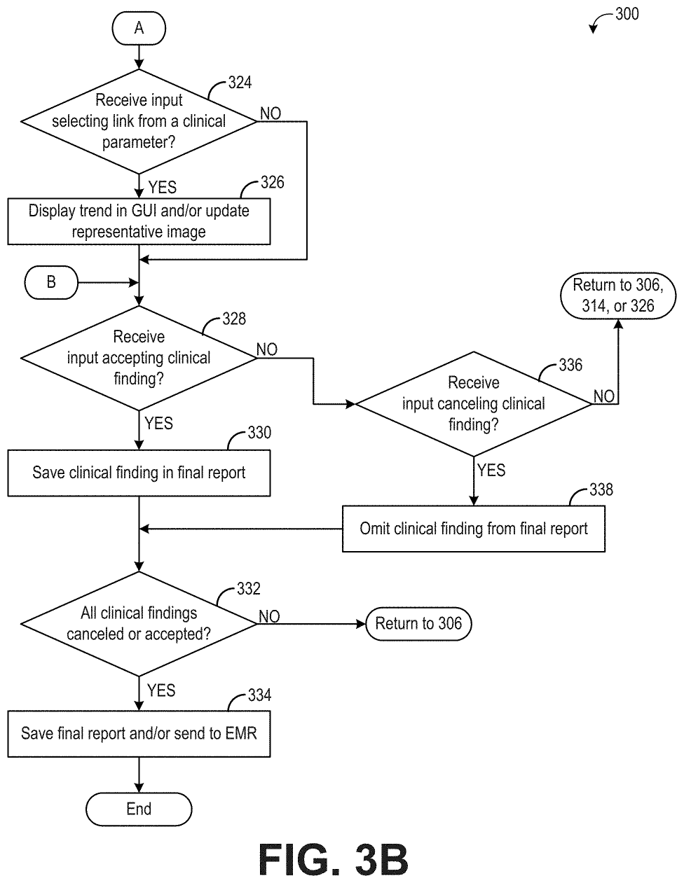

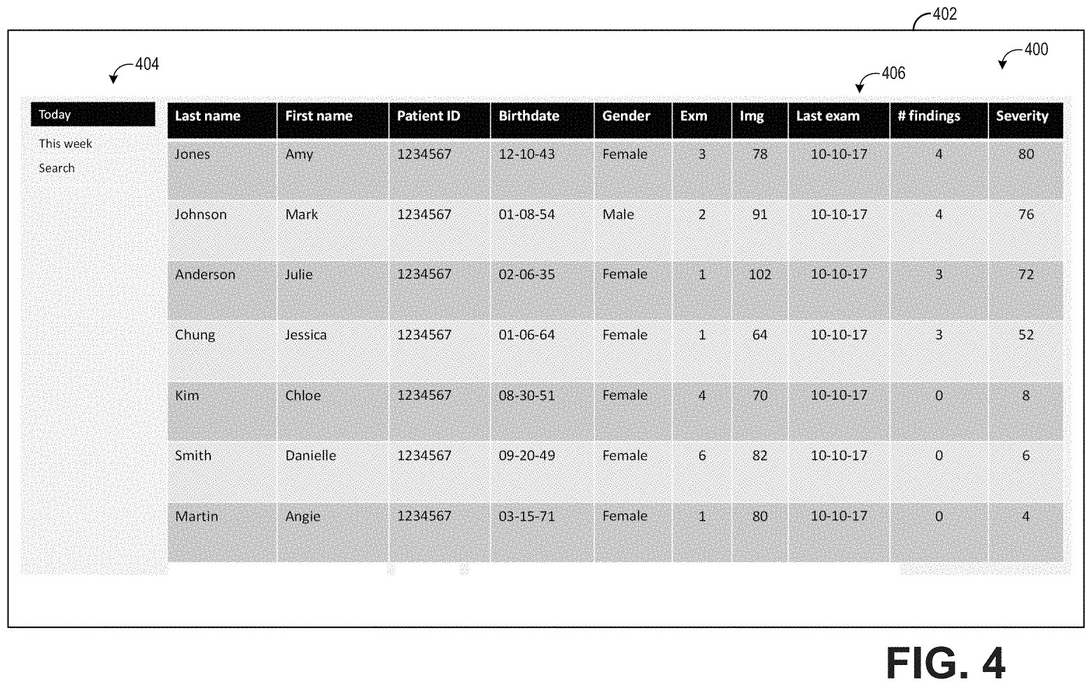

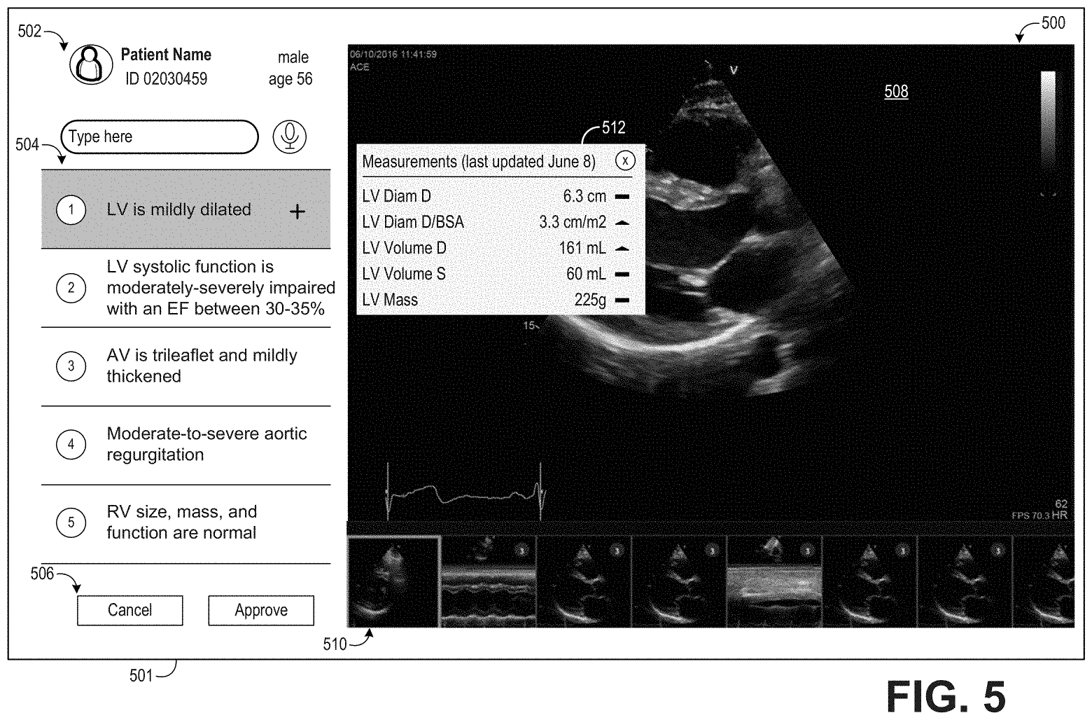

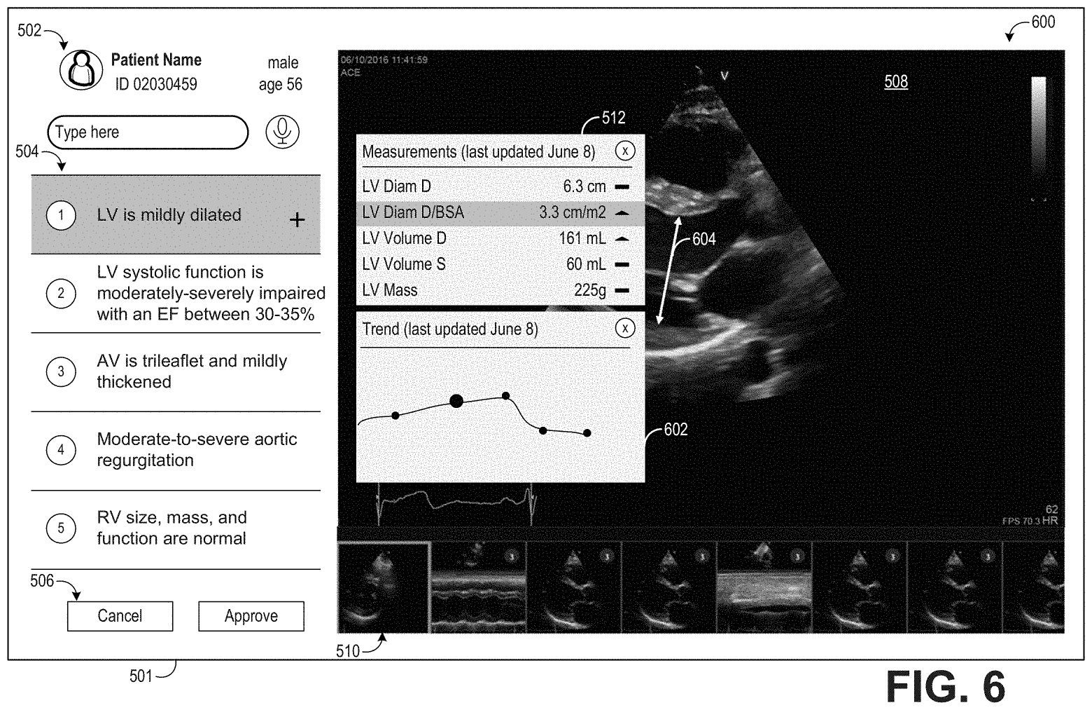

As mentioned above, the workflow according to the disclosure begins with the acquisition of a plurality of medical images using a suitable imaging modality, such as an ultrasound system as depicted in FIG. 1. The medical images may be sent to a suitable AI-enabled device (such as the server of FIG. 1) that is adapted to automatically generate a list of clinical findings from the medical images, according to the method illustrated in FIG. 2. Once an exam has been automatically interpreted by the AI-enabled device, various graphical user interfaces may be presented to a clinician. For example, according to the method illustrated in FIGS. 3A and 3B, a first GUI may be displayed that includes a plurality of patient exams sorted by severity of the clinical findings, as shown in FIG. 4. Further, a second GUI may be displayed that includes the clinical findings for a selected patient, as shown in FIG. 5. The clinician viewing the second GUI may navigate through the clinical findings and view the underlying measurements and/or images from which the clinical findings were based, as shown in FIG. 6. The clinician may then assemble a final report for the patient's medical record by accepting or canceling each clinical finding.

It should be appreciated that while the various embodiments are described herein with respect to ultrasound imaging, the methods described herein may be implemented with other imaging modalities, including but not limited to computed tomography (CT), magnetic resonance imaging (MRI), positron emission tomography (PET), single photon emission computed tomography (SPECT), and so on.

FIG. 1 illustrates an example imaging system 10. The imaging system 10 includes an imaging modality, herein in the form of an ultrasound imaging system 100, one or more servers 130, and one or more workstations 140. The system 10 may include any number of imaging modalities, servers 130, and workstations 140 and is not limited to the embodiment of system 10 illustrated in FIG. 1. The components of the system 10 may communicate via wired and/or wireless communication, for example, and may be separate systems and/or integrated to varying degrees, for example. Server 130 may include one or more devices of a Picture Archiving and Communication System (PACS) server and/or other medical information systems including but not limited to EMR systems, workflow systems, and reporting systems.

In operation, the imaging modality obtains one or more images of a patient anatomy. The imaging modality may include any device capable of capturing an image of a patient anatomy such as a medical diagnostic imaging device. For example, the imaging modality may include an X-ray imager, ultrasound scanner, magnetic resonance imager, or the like.

An example imaging modality in the form of ultrasound imaging system 100 is described herein. The ultrasound imaging system 100 includes a transmit beamformer 101 and a transmitter 102 that drive transducer elements 104 within a probe 106 to emit pulsed ultrasonic signals into a body (not shown). A variety of geometries of probes and transducer elements may be used. The pulsed ultrasonic signals are back-scattered from structures in the body, such as blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. According to some embodiments, the probe 106 may contain electronic circuitry to do all or part of the transmit and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound system.

A user interface 115 may be used to control operation of the ultrasound imaging system 100, including controlling the input of patient data, changing a scanning or display parameter, and the like. The user interface 115 may include a graphical user interface configured for display on a display device 118. The graphical user interface may include information to be output to a user (such as ultrasound images, patient data, etc.) and may also include menus or other elements through which a user may enter input to the computing system. The user interface 115 may include one or more of the following: a rotary, a mouse, a keyboard, a trackball, a touch-sensitive display, hard keys linked to specific actions, soft keys that may be configured to control different functions, and a graphical user interface.

The ultrasound imaging system 100 also includes a processor 116 to control the transmit beamformer 101, the transmitter 102, the receiver 108, and the receive beamformer 110. The processor 116 is in electronic communication with the probe 106. For the purposes of this disclosure, the term “electronic communication” may be defined to include both wired and wireless communications. The processor 116 may control the probe 106 to acquire data. The processor 116 controls which of the elements 104 are active and the shape of a beam emitted from the probe 106. The processor 116 is also in electronic communication with a display device 118, and the processor 116 may process the data into images for display on the display device 118. The processor 116 may include a central processor (CPU) according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment, the demodulation can be carried out earlier in the processing chain.

The processor 116 is adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. For example, an embodiment may acquire images at a real-time rate of 7-20 volumes/sec. The ultrasound imaging system 100 may acquire 2D data of one or more planes at a significantly faster rate. However, it should be understood that the real-time volume-rate may be dependent on the length of time that it takes to acquire each volume of data for display. Accordingly, when acquiring a relatively large volume of data, the real-time volume-rate may be slower. Thus, some embodiments may have real-time volume-rates that are considerably faster than 20 volumes/sec while other embodiments may have real-time volume-rates slower than 7 volumes/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a volume-rate of, for example, 10 Hz to 100 Hz. Images generated from the display may be refreshed at a similar frame-rate. Other embodiments may acquire and display data at different rates. For example, some embodiments may acquire data at a volume-rate of less than 10 Hz or greater than 30 Hz depending on the size of the volume and the intended application. A memory 120 is included for storing processed volumes of acquired data. In an exemplary embodiment, the memory 120 is of sufficient capacity to store at least several seconds' worth of volumes of ultrasound data. The volumes of data are stored in a manner to facilitate retrieval thereof according to its order or time of acquisition. The memory 120 may comprise any known data storage medium.

Optionally, embodiments of the present disclosure may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present disclosure, data may be processed by other or different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. The image beams and/or volumes are stored and timing information indicating a time at which the data was acquired in memory may be recorded. The modules may include, for example, a scan conversion module to perform scan conversion operations to convert the image volumes from beam space coordinates to display space coordinates. A video processor module may be provided that reads the image volumes from a memory and displays an image in real time while a procedure is being carried out on a patient. A video processor module may store the images in the memory 120, from which the images are read and displayed.

In an embodiment, the processor 116 may apply one or more preprocessing functions, for example, to the image data in order to prepare the image for viewing on a display. For example, the processor 116 may convert raw image data into a DICOM standard format or attach a DICOM header. Preprocessing functions may include modality-specific enhancements, for example (e.g., contrast or frequency compensation functions specific to a particular X-ray imaging device), applied at the beginning of an imaging and display workflow. The preprocessing functions differ from processing functions applied to image data in that the processing functions are not modality specific and are instead applied at the end of the imaging and display workflow (for example, at a display). However, in some examples, the preprocessing functions may be performed by a separate device, such as a workstation 140 or server(s) 130.

The image data (e.g., unprocessed image data or image data that has been processed into images) may be communicated between the processor 116/memory 120 and one or more remote devices, such as the server(s) 130 and/or one or more workstations 140. The image data may be communicated electronically over a wired or wireless connection, for example.

The server(s) 130 may include one or more computing devices including computer-readable storage media suitable for storing the image data (e.g., sent from ultrasound imaging system 100) for later retrieval and viewing at a workstation 140. The server(s) 130 may also include one or more software applications for additional processing and/or preprocessing of the image data by one or more workstations 140. Server(s) 130 may include resources (e.g., memory 134, processor(s) 132) that may be allocated to store and execute an AI-based virtual parameter assistant 138 and an AI-based virtual diagnostic assistant 139. The virtual assistants may be realized as separate assistants each for a different purpose, as described herein, or as one overall virtual assistant, which represents all the different attributes that will be hereby elaborated. In other words, one AI-based virtual assistant may be executed on server(s) 130, to both generate clinical parameters and identify clinical findings.

Virtual parameter assistant 138 is configured to analyze images to identify anatomical features present within the images and identify clinical parameters of the anatomical features. For example, virtual parameter assistant 138 may analyze a set of images of a patient's heart acquired with the ultrasound imaging system 100 and identify anatomical features of the heart within each image of the set of images, including the left ventricle, right ventricle, left atrium, right atrium, aorta, etc. Each image of the set of images may be tagged with each anatomical feature identified in that image. Virtual parameter assistant 138 is configured to generate a plurality of clinical parameters relating to the identified anatomical features as indicated by a diagnostic workflow protocol. The plurality of clinical parameters may include quantitative measurements of structural features of the anatomical features, such as length/width measurements, volume measurements, and mass estimates. The plurality of clinical parameters may further include functional analysis of certain anatomical features, also referred to as physiological features, such as flow rate measurements, flow direction measurements, time measurements (e.g., duration that a heart valve is open in a given pumping cycle), and so forth. Thus, while description provided herein generally relates to measurements performed on single images, is to be understood that some measurements may be made across multiple frames of imaging data. Further, some measurements may include other medical data in addition to the imaging data, such as electrocardiogram data.

For example, virtual parameter assistant 138 may be trained to measure and/or identify a plurality of clinical parameters typically measured during an echocardiogram, such as left ventricle diameter, left ventricle volume, aortic regurgitation, and so forth. Accordingly, virtual parameter assistant 138 may retrieve each image that includes a particular anatomical feature under consideration, such as a left ventricle, and identify one or more clinical features of that anatomical feature, such as by identifying boundaries of the left ventricle and measuring the diameter of the left ventricle by determining the distance from a top boundary to a bottom boundary of the ventricle. Virtual parameter assistant 138 may repeat that process until each clinical parameter defined in the clinical workflow protocol has been measured and/or identified.

The measurements and/or functional analysis results generated by virtual parameter assistant 138 may be stored along with the set of images (and any other relevant medical data obtained during or around the imaging sessions, such as electrocardiogram data) at server(s) 130. Where appropriate, one or more images may be annotated with relevant measurements. For example, an image including a view of a left ventricle may be annotated to include an indication of the measurement of the diameter of the left ventricle, such as in the form of a line with end points intersecting each relevant boundary of the left ventricle.

In examples where more than one image is present that includes an anatomical feature under consideration where a given measurement may be performed, virtual parameter assistant 138 may select a best image (e.g., based on signal to noise ratio, view angle, or other parameter) and perform a single measurement using the best image. In another example, virtual parameter assistant 138 may perform a respective measurement on two or more images that include the anatomical feature under consideration, and generate a final measurement by averaging the measurements. In a still further example, the virtual parameter assistant 138 may perform a respective measurement on two or more images that include the anatomical feature under consideration, and generate a list of measurements that may be ranked based on a confidence level of each measurement. The confidence level may be based on the signal to noise ratio of the image, image sharpness, or other parameters that may affect how accurately virtual parameter assistant 138 was able to measure the given clinical parameter.

Virtual diagnostic assistant 139 is configured to generate a list of clinically relevant findings based on the clinical parameters (e.g., measurements and functional analyses) generated by virtual parameter assistant 138, the clinical workflow, and clinical guidelines. For example, the clinical workflow may inform virtual diagnostic assistant 139 of the potential clinical findings to generate, and virtual diagnostic assistant 139 may identify clinical findings by comparing the clinical parameters generated by virtual parameter assistant 138 to various guidelines, which may include normal ranges of the clinical parameters obtained from published guidelines, research studies, etc. In some examples, the normal ranges for the clinical parameters may be adjusted based on patient information (e.g., patient gender, patient age). In other examples, virtual diagnostic assistant 139 may be trained to generate only specific findings for specific patients (e.g., virtual diagnostic assistant 139 may be trained to only generate clinical findings for echocardiograms of adult men).

Thus, as an example, virtual diagnostic assistant 139 may generate a list of clinical findings for an echocardiogram by analyzing the list of clinical parameters generated by virtual parameter assistant 138 and comparing each clinical parameter to an associated guideline. For example, referring to the left ventricle diastolic diameter, virtual diagnostic assistant 139 may compare the measurement of the left ventricle diameter during diastole generated by virtual parameter assistant 138 (e.g., 6.3 cm) to a normal range of left ventricle diastolic diameters for men (e.g., 4.2-5.9 cm) and generate a clinical finding that the left ventricle diastolic diameter is larger than normal. Further, virtual diagnostic assistant 139 may determine a level of severity of the finding, e.g., the virtual diagnostic assistant 139 may determine that a left ventricle diastolic diameter of 6.3 cm is mildly abnormal (e.g., as opposed to normal, moderately abnormal, and severely abnormal).

The list of clinical findings generated by virtual diagnostic assistant 139 may include abnormal clinical findings, as described above. However, the list of clinical findings generated by virtual diagnostic assistant 139 may also include normal findings. For example, if the left ventricle diastolic diameter is determined by virtual parameter assistant 138 to be 5.7 cm, virtual diagnostic assistant 139 may determine that the left ventricle diastolic diameter is normal and include the normal indication in the list of clinical findings.

Some clinical findings may be based solely on one clinical parameter, while other clinical findings may be based on two or more clinical parameters (e.g., a determination of an enlarged left ventricle may be based on left ventricle diastolic diameter, diastolic volume, systolic volume, and mass). In examples where a clinical finding is based on more than one clinical parameter, each clinical parameter that contributed to the clinical finding may be tagged and/or otherwise identified and saved together as a list of determinative clinical parameters. Each determinative clinical parameter may be given a weighted confidence score, at least in some examples. The weighted confidence score may be similar to the confidence score given to each clinical parameter described above (e.g., based on image quality), and may be weighted based on a correlation between that clinical parameter and the clinical finding. When the list of determinative clinical parameters are presented to a clinician for review, the determinative clinical parameters may be sorted based on the weighted confidence scores, for example.

Virtual diagnostic assistant 139 may be configured to rank each clinical finding by severity or other parameter. For example, any clinical findings that are determined to be severely abnormal may be given a first, highest ranking (e.g., a score of 3), any clinical findings that are determined to be moderately abnormal may be given a second ranking (e.g., a score of 2), any clinical findings that are determined to be mildly abnormal may be given a third ranking (e.g., a score of 1), and any clinical findings that are determined to be normal may be given a fourth, lowest ranking (e.g., a score of 0). The list of clinical findings may then be sorted based on ranking. Further, an overall severity score for a patient may be determined by summing the ranking scores of each clinical finding for that patient.

The virtual parameter assistant and virtual diagnostic assistant may be trained using machine learning (e.g., deep learning), such as neural networking or other training mechanisms that are specific to diagnostics based on medical imaging. The virtual assistants may be trained in a suitable manner. For example, virtual parameter assistant 138 may be trained to take measurements typically associated with an echocardiogram. The training may include supplying a set of medical images of human hearts, in views typically obtained during an echocardiogram, to virtual parameter assistant 138. Each image may be tagged (in a format readable by the virtual parameter assistant) with the anatomical and physiological features in the image and clinical parameters of the anatomical and physiological features that are to be measured, generated, and/or identified. The definitions of the clinical parameters may include locations of endpoints for distance measurements, perimeter traces for area measurements, 3D segmentation for volume measurements (e.g., generating a 3D mesh and classifying voxels in 3D), equations for calculating volume and mass measurements, changes in anatomical features over time, and so forth. Virtual parameter assistant 138 may then learn to identify anatomical features in patient medical images and generate the clinical parameters.

Virtual diagnostic assistant 139 may be trained in a similar manner. For example, virtual diagnostic assistant 139 may be supplied with a set of lists of clinical parameters typically obtained during an echocardiogram and clinical findings associated with each list of clinical parameters, as well as normal ranges for each clinical parameter. Virtual diagnostic assistant 139 may then learn to identify clinical findings based on a list of clinical parameters, e.g., as supplied by virtual parameter assistant 138. Further, the training of the virtual assistants may include data-driven training concepts as described above, and/or may include model-driven training concepts (e.g., where a mathematical 3D model of the anatomy of interest is used to provide the automated measurements).

Server(s) 130 includes a communication module 136, memory 134, and processor(s) 132 to store and execute the virtual parameter assistant 138 and virtual diagnostic assistant 139, as well as send and receive communications and other information, generate and output graphical user interfaces, store images, generate and output final reports upon clinician review of the automatically determined clinical findings, and other actions.

Communication module 136 facilitates transmission of electronic data within and/or among one or more systems. Communication via communication module 136 can be implemented using one or more protocols. In some examples, communication via communication module 136 occurs according to one or more standards (e.g., Digital Imaging and Communications in Medicine (DICOM), Health Level Seven (HL7), ANSI X12N, etc.). Communication module 136 can be a wired interface (e.g., a data bus, a Universal Serial Bus (USB) connection, etc.) and/or a wireless interface (e.g., radio frequency, infrared, near field communication (NFC), etc.). For example, communication module 136 may communicate via wired local area network (LAN), wireless LAN, wide area network (WAN), etc. using any past, present, or future communication protocol (e.g., BLUETOOTH™, USB 2.0, USB 3.0, etc.).

Memory 134 includes one or more data storage structures, such as optical memory devices, magnetic memory devices, or solid-state memory devices, for storing programs and routines executed by processor(s) 132 to carry out various functionalities disclosed herein. Memory 134 may include any desired type of volatile and/or non-volatile memory such as, for example, static random access memory (SRAM), dynamic random access memory (DRAM), flash memory, read-only memory (ROM), etc. Processor(s) 132 may be any suitable processor, processing unit, or microprocessor, for example. Processor(s) 132 may be a multi-processor system, and, thus, may include one or more additional processors that are identical or similar to each other and that are communicatively coupled via an interconnection bus.

One or more of the devices described herein may be implemented over a cloud or other computer network. In certain examples, users (e.g., a cardiologist or other clinician) can access functionality provided by server(s) 130 via a software-as-a-service (SaaS) implementation over a cloud or other computer network, for example. In certain examples, all or part of server(s) 130 can also be provided via platform as a service (PaaS), infrastructure as a service (IaaS), etc. For example, server(s) 130 can be implemented as a cloud-delivered Mobile Computing Integration Platform as a Service. A set of consumer-facing Web-based, mobile, and/or other applications enable users to interact with the PaaS, for example. Further, while server(s) 130 is shown in FIG. 1 as constituting a single entity, it is to be understood that server 130 may be distributed across multiple devices, such as across multiple servers and/or the cloud.

While not specifically shown in FIG. 1, additional devices described herein (such as workstations 140) may likewise include user input devices, memory, processors, and communication modules/interfaces similar to communication module 136, memory 134, and processor(s) 132 described above, and thus the description of communication module 136, memory 134, and processor(s) 132 likewise applies to the other devices described herein.

As shown in FIG. 1, a plurality of workstations 140 may be communicatively coupled to server(s) 130. Each workstation may include a processor, memory, communication module, user input device, display (e.g., screen or monitor), and/or other subsystems and may be in the form of a desktop computing device, a laptop computing device, a tablet, a smart phone, or other device. Each workstation may be adapted to send and receive encrypted data, display medical information, including medical images in a suitable format such as digital imaging and communications in medicine (DICOM) or other standards, and ensure only authenticated users have access to patient medical information. The workstations may be located locally at the same medical facility as the imaging modality and/or remotely from the medical facility (such as a care provider's mobile device).

The workstations 140 may retrieve or receive image data from the server(s) 130 for display to one or more users. For example, a workstation 140 may retrieve or receive graphical user interfaces in order to display clinical findings of a patient as determined by the virtual diagnostic assistant, image data representative of the diagnostic exam annotated with measurements taken by the virtual parameter assistant, and so forth. In some examples, server(s) 130 may send the graphical user interfaces to the workstation for display. In other examples, the output of the virtual parameter assistant and virtual diagnostic assistant may be sent to the workstation 140 (including relevant medical images), and the workstation may generate the graphical user interfaces for display. In some examples, user interface templates may be stored in memory of the workstation 140, with pointers that may be used to retrieve relevant information from server(s) 130. A user may then examine the images and measurements to determine whether the clinical findings are accurate. The clinical findings confirmed as accurate by the user may then be included in a report and sent to the patient's medical record, which may be stored in an electronic medical record database on server(s) 130 or external to server(s) 130.

FIG. 2 shows a flow chart illustrating an example method 200 for generating a list of clinical findings from diagnostic medical images of a patient. Method 200 will be described herein with reference to the system and components depicted in FIG. 1, though it should be understood that the method may be applied to other systems and components without departing from the scope of the present disclosure. Method 200 may be carried out by a computing device, such as server(s) 130, and may be stored as executable instructions in non-transitory memory of the computing device (e.g., server(s) 130).

At 202, method 200 includes receiving a request to analyze patient images. In an example, as indicated at 204, receiving the request to analyze patient images may include receiving a request to analyze patient images received from a workstation (or other computing device) associated with an imaging modality. The imaging modality may include the ultrasound imaging system 100 of FIG. 1, or other suitable modality. The patient images may include standard 2D images, and may additionally or alternatively include 3D images, Doppler ultrasound information, videos, or other suitable information obtained from the imaging modality. In one example, when the images are received, the analysis of the images may commence immediately once processing resources (e.g., on the server(s) 130) are available, and thus the receipt of the images may serve as the request to analyze the images. The images that are received from the imaging modality may be stored on the computing device (e.g., server(s) 130). Further, as indicated at 206, receiving the request to analyze the patient images may include receiving a diagnostic workflow protocol, clinician information, and/or patient information. In this way, the images saved on the server(s) 130 may be tagged with the diagnostic workflow protocol that is to be carried out (e.g., echocardiogram), referring clinician (e.g., the cardiologist who ordered the echocardiogram and/or the cardiologist who will review the results of the echocardiogram), and patient information (e.g., name, date of birth, gender).

The patient information may further include relevant medical history of the patient. For example, if the patient has previously been diagnosed with a condition related to the diagnostic workflow protocol, the diagnosed condition may be included in the patient information. As an example, if the diagnostic workflow protocol is an echocardiogram and the patient was previously diagnosed with cardiomyopathy, the cardiomyopathy may be included in the patient information. Additional relevant medical history may include if the same or related type of exam (e.g., same diagnostic workflow protocol) had previously been conducted on the patient, for example if an echocardiogram had previously been conducted. If the same exam (or a related exam where relevant diagnostic information may have been determined) has been previously conducted on the patient, the patient information may include some or all of the clinical findings of the previous exam.

At 208, method 200 includes automatically generating a set of clinical parameters relating to selected patient features as defined by the diagnostic workflow protocol. The list of clinical parameters may be generated by an artificial intelligence-based parameter assistant executed on the computing device, such as virtual parameter assistant 138 executed on server(s) 130. As explained above with respect to FIG. 1, the AI-based parameter assistant may generate/determine the set of clinical parameters according to a clinical parameter algorithm that measures/identifies various clinical parameters in the received images. The clinical parameter algorithm executed by the AI-based parameter assistant may be an algorithm/model trained by deep learning or other suitable mechanism. For example, to automatically identify clinical parameters in echocardiogram images, the AI-based parameter assistant may be trained with a plurality of prior echocardiogram images that have been annotated or tagged with the anatomical and/or physiological features present in each image, and corresponding measurement, classification, and/or score of each relevant anatomical and/or physiological feature. Further, the AI-based parameter assistant may continue to learn new clinical parameters and/or refine identification of learned clinical parameters with each exam that is performed.

To generate the set of clinical parameters, the patient images are analyzed and the anatomical features and/or physiological features present in each image are identified, which may include the view angle or cross-section of the images (e.g., parasternal long-axis view, apical four chamber view). The anatomical features may include the various chambers of the heart, the various valves of the heart, and the pericardium, for example, and may further include the anatomical features at different stages of a pumping cycle (e.g., diastolic versus systolic). The physiological features may include aspects of how the anatomical features are functioning, and thus identifying physiological features in the images may include identifying images or sets of images where parameters such as blood flow rate and/or direction across the valves, motion of one or more anatomical features, etc., may be measured.

Once the anatomical and/or physiological features are identified, the clinical parameters defined by the diagnostic workflow protocol are measured or determined. The clinical parameters may include quantitative measurements, classifications, and/or scores, as indicated at 210. The quantitative measurements may include dimensions of the anatomical features that may be measured from the images, such as length/diameter, volume, and mass of various anatomical features, and may also include measurements of functional features that define how an anatomical structure is performing, such as measurements of blood flow rate, ejection fraction, elapsed time for a given action to occur (e.g., mitral valve deceleration), pressure measurements, strain/force measurements, and so forth. The classifications may include the overall shape or appearance of an anatomical feature, which may be determined by the AI-based parameter assistant by performing object recognition, brightness detection, or other suitable analysis on the images. For example, the classifications may include an indication of whether certain abnormal or determinative features are present, such as presence of a ruptured chordae, an abnormal growth, or bulging septum. The scores may include a numerical value being assigned to various levels or stages of degradation or changes in anatomical or physiological features, and may include a severity score/index for mitral regurgitation, a calcium score, etc. Additionally, the clinical parameters may include index or ratio values, whereby one or more automatically determined clinical parameters are presented as an index or ratio of another parameter in order to normalize the clinical parameter. For example, left ventricle diastolic diameter may be presented as an absolute value as well as a normalized (e.g., index value) based on patient body surface area, body mass index, or other suitable indicator of body size, mass, organ size, metabolic function, etc. The additional parameters used in the index values may be obtained from the patient medical record, entered via user input, or other suitable mechanism.

Thus, the set of clinical parameters may include suitable parameters that may be used to determine if any abnormal clinical findings are present in the patient images, and therefore depend on the particular diagnostic workflow protocol being carried out, as indicated at 212. For example, the diagnostic workflow protocol may define which clinical parameters are to be measured, determined, and/or identified on which anatomical and/or physiological features. Using the echocardiogram as an example, the diagnostic workflow protocol may define which distance, volume, mass, etc., measurements are to be taken on various anatomical features, such as the left ventricle, right ventricle, aorta, etc. The clinical parameters included in an echocardiogram workflow may be different than the clinical parameters included in a fetal ultrasound workflow, for example. In some examples, the diagnostic workflow protocol may include instructions that the AI-based parameter assistant may follow to obtain the clinical parameters. In other examples, the diagnostic workflow protocol may direct the computing device to select an appropriate AI-based parameter assistant to generate the clinical parameters. For example, multiple different virtual parameter assistants may be stored on and/or executable by server(s) 130, with each virtual parameter assistant trained to generate clinical parameters for a single specific diagnostic protocol (e.g., echocardiogram, fetal ultrasound, brain scan). The diagnostic workflow protocol may identify which virtual parameter assistant is to be used to generate the clinical parameters.

The set of clinical parameters may be saved in memory (e.g., of server(s) 130) and associated with the patient images. Additionally, as indicated at 214, selected images may be annotated with representations of one or more clinical parameters. For example, an image showing a cross-sectional view of the left ventricle during diastole may be annotated with a line drawn between the points used to measure the left ventricle diastolic diameter. As another example, an image showing the presence of a ruptured chordae may be annotated with a line pointing to the ruptured chordae or painted with a color or box in the region of the ruptured chordae.

At 216, method 200 includes automatically identifying one or more clinical findings based on the clinical parameters (e.g., as determined at 208), stored guidelines, and patient information. The clinical findings may be identified by an AI-based diagnostic assistant executed on server(s) 130, such as virtual diagnostic assistant 139. As explained above with respect to FIG. 1, the AI-based diagnostic assistant may identify the clinical findings according to a clinical finding algorithm that compares the set of clinical parameters generated by the AI-based parameter assistant to guidelines (e.g., normal and abnormal ranges for each clinical parameter) in order to classify each clinical parameter as normal or abnormal, and further to identify a plurality of clinical findings based on respective determinative clinical parameters. The clinical finding algorithm executed by the AI-based diagnostic assistant may be an algorithm/model trained by deep learning or other suitable mechanism. For example, to automatically identify clinical findings in an echocardiogram exam, the AI-based diagnostic assistant may be trained with a plurality of prior sets of echocardiogram clinical parameters that have been annotated or tagged with the associated clinical findings. Further, the AI-based diagnostic assistant may continue to learn new clinical findings and/or refine identification of learned clinical findings with each exam that is performed.

To identify the one or more clinical findings, the clinical parameters determined at 208 may be compared to respective guidelines that define whether a given clinical parameter is normal or abnormal, and if abnormal, the degree of severity of the abnormality. The guidelines may be selected based on patient gender, age, and/or other factors. Each clinical parameter may optionally be assigned a confidence score, as indicated at 218. The confidence score may indicate how accurate the determination of the normal or abnormal determination is, for example based on image quality or other factors that may affect the measurement of the clinical parameter. For example, if the boundaries of the left ventricle are not sufficiently sharp, the measurement of the ventricle diameter may be given a low confidence score relative to a higher confidence score when the boundaries are relatively sharp.

As indicated at 220, the clinical findings may be identified based on determinative clinical parameters (where the determinative clinical parameters may be identified based on the stored guidelines), and optionally based patient information and/or on the confidence scores. The AI-based diagnostic assistant may be trained to classify the clinical parameters based on which clinical finding(s) each clinical parameter is associated with (e.g., which clinical parameters are determined to be indicative of which clinical findings). For example, a clinical finding of impaired left ventricle systolic function may be made on the basis of ejection fraction alone, particularly if the ejection fraction is below 40%. By comparing the ejection fraction to a normal range of ejection fractions, a clinical finding of impaired left ventricle systolic function may be identified when the ejection fraction is less than 50% (where the normal range of ejection fractions includes 50-75%), and the level of severity of the impairment may be identified based on how much below the normal range the patient's ejection fraction is (e.g., 40-49% may be mildly impaired, 35-39% may be moderately impaired, and below 35% may be severely impaired).

Other clinical findings may be based on multiple clinical parameters. For example, a clinical finding of diastolic dysfunction (grade I, II or III) may be based on four different measurements made in four or five different images. For example, a clinical finding of grade II diastolic dysfunction may be based on the parameters E (early filling), A (atrial filling), e′ (early diastolic velocity), TRmax (maximum tricuspid regurgitation), and LAvol (left atrium volume), and the relevant images from which these clinical parameters are determined may include one or more tissue velocity imaging (TVI) images, one or more mitral Doppler images, and one or more B-mode in LA images. Still further, a given clinical parameter may be used to determine more than one clinical finding.

In one non-limiting example, the determinative clinical parameter(s) on which a given clinical finding is identified may be tagged, aggregated, or otherwise listed together, for ultimate presentation to a clinician. To further expedite clinician review of the clinical finding, the determinative clinical parameters may be sorted based on the confidence scores assigned to the clinical parameters, and may further be sorted based on a correlation between the clinical finding and the clinical parameter. For example, a first clinical parameter highly correlated with a clinical finding and also having a high confidence score may be given a first, higher weight. A second clinical parameter weakly correlated with the clinical finding and also having a low confidence score may be given a second, lower weight. A third clinical parameter highly correlated with the clinical finding but having a low confidence score may be given a third, intermediate weight. Such a configuration may allow the reviewing clinician additional insight into how the automated system (e.g., virtual diagnostic assistant) identified the clinical finding.

Additionally, at least in some examples, the clinical findings may be further identified and/or defined based on patient information. The patient information may be determined via non-automated mechanisms, at least in some examples, such as from user input, a patient medical record (e.g., obtained from a remote computing device operably coupled to the computing device), or other suitable source, and may include medical history (e.g., past diagnoses), demographic information, and/or other relevant clinician observations. For example, if the patient information indicates that the patient has a prior diagnosis of dilated cardiomyopathy, the clinical parameters leading to the original diagnosis of the dilated cardiomyopathy (e.g., left ventricle diameter and volume, heart wall thickness, etc.) may be compared to the current clinical parameters to determine if the cardiomyopathy has increased in severity, spread to other chambers of the heart, or stabilized. Further, due to the prior indication of cardiomyopathy, a different algorithm may be used to determine the clinical findings, for example an algorithm that weights the clinical parameters differently (e.g., more or less stringent) when correlating the clinical parameters with a clinical finding.

At 222, method 200 includes assigning the patient a severity score based on the identified clinical findings. The severity score may be based on the type of clinical findings (e.g., impaired left ventricle systolic function may be assigned a higher severity score than a dilated left ventricle) as well as a level of severity of the clinical finding (e.g., a mildly dilated left ventricle may be assigned a lower severity score than a highly dilated left ventricle). Normal clinical findings may be assigned a severity score of zero, at least in some examples. The severity scores for all the clinical findings for the patient may be summed to determine an overall patient severity score.

At 224, method 200 includes storing the clinical findings, clinical parameters, and patient severity score with the annotated images as an exam for review, and tagging the exam with the patient name, clinician name, and diagnostic protocol workflow. At 226, method 200 determines if additional exams (e.g., for other patients) are ready to be analyzed. If yes, method 200 loops back to 202 to analyze medical images of a subsequent patient in order to identify the clinical findings for the subsequent patient. If no, method 200 proceeds to 228 to sort some or all of the completed exams by clinician, severity score, and/or other factors such as diagnostic workflow protocol, date, etc. For example, all echocardiograms ordered by (or to be reviewed by) a first cardiologist over a given duration (e.g., day or week) may be sorted based on patient severity score. As will be explained in more detail below, the completed exams may be reviewed by a clinician in order to confirm or cancel each clinical finding and assemble a final report to be saved in the patient's medical record. By sorting the exams by date and severity score, for example, the clinician may be given an overview of the scope of the exams to be reviewed, allowing the clinician to prioritize review of certain exams (e.g., exams with high patient severity scores, where expedited diagnosis and treatment may be desired) over other exams (e.g., exams with low patient severity scores, where immediate treatment may not be indicated). However, the above-described embodiment of waiting until all exams have been analyzed before sorting the exams is non-limiting and other configurations are possible. For example, each time an exam is completed, that exam may be placed into a list of sorted exams based on date and severity score, for example. In other examples, the exams may be binned by reviewing clinician, but otherwise not sorted. Other configurations are possible. Method 200 then returns.

It should be appreciated that although the method 200 is described primarily with regard to patient images acquired via ultrasound imaging, method 200 may be implemented with images acquired other imaging modalities, including but not limited to computed tomography (CT), magnetic resonance imaging (MM), positron emission tomography (PET), single photon emission computed tomography (SPECT), and so on. It should be further appreciated that although method 200 is described primarily with respect to echocardiograms, method 200 may be implemented using other diagnostic workflow protocols, including but not limited to exams of other organs (e.g., liver, brain), whole body exams, functional exams, and so forth.

FIGS. 3A and 3B illustrate a method 300 for displaying the results of an automatically-performed diagnostic analysis of patient medical images. Method 300 will be described herein with reference to the system and components depicted in FIG. 1, though it should be understood that the method may be applied to other systems and components without departing from the scope of the present disclosure. Method 300 may be carried out by a computing device, such as server(s) 130 or workstation 140, and may be stored as executable instructions in non-transitory memory of the computing device (e.g., server(s) 130 or workstation 140).

At 302, method 300 includes outputting a first graphical user interface (GUI) that includes a plurality of sorted exams. The first GUI may be output in response to a request, such as a request from a user (e.g., a clinician may enter user input to the computing device requesting display of all completed exams for a given time duration). Each exam in the list of sorted of exams may be generated according to the method 200 described above with respect to FIG. 2.

FIG. 4 shows an example first graphical user interface 400 that may be displayed on a display device 402. Display device 402 may be included as part of, or otherwise communicatively coupled to, workstation 140 or server(s) 130. First GUI 400 includes a side bar 404 where various tabs are displayed. As shown, the tabs include “today,” “this week,” and “search.” A user may select a tab (e.g., via a mouse click, mouse hover, touch input, voice command, etc.) in order to view the exams associated with that tab, or to perform a search for a particular exam. As shown in FIG. 4, a user has selected the “today” tab, resulting in display of a list of completed exams 406, where the completed exams have been performed, analyzed, and/or are ready for review that day. Each exam may be identified by patient name and other patient information. Further, select information regarding each exam, including number of images in the exam, exam date, and number abnormal findings, may also be displayed. The exams are sorted by patient severity score, although other mechanisms for organizing the exams are possible.

Returning to FIG. 3A, method 300 includes determining if a request to display a patient specific, second graphical user interface has been received. As an example, the user viewing the first graphical user interface (e.g., first GUI 400 of FIG. 4) may select a particular exam by entering an appropriate input (e.g., mouse click on a particular exam, touch input on a particular exam, etc.). If a request to display a patient specific GUI has not been received, method 300 continues to output/display the first GUI. If a request to a view a patient specific GUI is received, method 300 proceeds to 306 to output the patient GUI in a first configuration for display. The patient GUI in the first configuration may include a visualization of a listing of clinical findings for the selected patient exam, as indicated at 308. In some examples, the clinical findings may be sorted by severity and/or confidence. The clinical findings may include only abnormal findings in one example. In another example, the clinical findings may include both abnormal findings and normal findings, if any abnormal findings are present. When both normal and abnormal findings are present, the clinical findings may be sorted so that the abnormal findings are presented first/at the top of the list, or otherwise highlighted relative to the normal findings. The patient GUI in the first configuration may further include each visualization of each clinical finding including a link to more information, as indicated at 310. As will be explained in more detail below, the additional information includes the determinative clinical parameter(s) from which that clinical finding was identified, one or more patient images relevant to the clinical finding, representations of trends of that clinical finding and/or determinative clinical parameters, and/or other information. Additionally, in some examples, the patient GUI in the first configuration may include a visualization of reference(s) to a prior exam, as indicated at 311. For example, a clinical finding may be compared to a prior clinical finding for that patient. As an example, a given clinical finding may include “when compared with exam of 01August-2016, no significant change was found” or “when compared with exam of 01-August-2016, severe aortic stenosis has replaced moderate aortic stenosis.” By including the reference to prior exams, ongoing or worsening conditions may be given additional relevance and context, facilitating fast clinician review.

Method 300 includes, at 312, determining if an input selecting a link within a clinical finding has been received. For example, as explained above with respect to FIG. 4, a clinical finding may include a link to additional information, which the user may select via mouse input, touch input, hovering over the clinical finding, etc. If a user input selecting the link is not received, method 300 proceeds to 328, which is shown in FIG. 3B and described in more detail below. If user input selecting the link is received, method 300 proceeds to 314 to output the patient GUI in a second configuration. The patient GUI in the second configuration may be similar to the patient GUI in the first configuration, but may include additional information superimposed on the existing displayed information, or otherwise presented within the patient GUI.

The patient GUI in the second configuration may include a visualization of a listing of determinative clinical parameters, as indicated at 316. The determinative clinical parameters may be the clinical parameters identified by the AI-based diagnostic module as contributing to the identification of the clinical finding (e.g., the particular clinical finding selected by the user). In some examples, the determinative clinical parameters may be sorted by relevance and/or confidence, as explained above. In still further examples, the user may provide input instructing the computing device how to sort the determinative clinical parameters. For example, if the determinative clinical parameters are sorted by relevance, the user may choose to instead view the clinical parameters sorted by confidence scores, or vice versa. Further, the user may choose to view the determinative clinical parameters by both relevance and confidence. In other examples, the determinative clinical parameters may be listed in no particular order.

As indicated at 318, at least some of the visualizations of the clinical parameters may each include a link to an annotated image and/or a trend line or graph related to that clinical parameter. The annotated image may include an image (or set of images) from which that clinical parameter was determined. The trend line or graph may include past determinations of that clinical parameter for that patient, where available. Other information may be presented, additionally or alternatively, such as normal and abnormal ranges for the clinical parameters, links to stored guidelines to assist in the clinician understanding or confirming presented clinical findings, etc.

The patient GUI in the second configuration may further include a representative image, as indicated at 320, and/or a plurality of thumbnail images, as indicated at 322. The representative image may include an anatomical and/or physiological feature related to the selected clinical finding, at least in some examples. For example, if the clinical finding is related to the left ventricle, and image including the left ventricle may be selected as the representative image. Likewise, the thumbnail images may be related to the selected clinical finding, e.g., each thumbnail image may include the left ventricle or be an image from which at least one of the determinative clinical parameters for the selected clinical finding was generated.

FIG. 5 shows an example patient graphical user interface in a second configuration 500 that may be displayed on a display device 501. Display device 501 may be included as part of, or otherwise communicatively coupled to, workstation 140 or server(s) 130. Patient GUI in the second configuration 500 includes patient information 502, including patient name, patient ID number, and patient demographic information (e.g., age and gender). Patient GUI in the second configuration 500 further includes a listing of clinical findings 504. In the example GUI shown in FIG. 5, the list of clinical findings includes five clinical findings, four of which are abnormal and one of which is normal. In some examples, only abnormal findings may be shown, while normal findings may be presented in a different GUI and/or in response to a user request. Patient GUI in the second configuration 500 further includes user interface control buttons 506, including a cancel button, an accept button, a search bar, and a voice command button. In some examples, initially the patient GUI may be displayed in the first configuration described above, where only the list of clinical findings and user interface control buttons are displayed, and patient GUI in the second configuration 500 may only be displayed responsive to selection of one of the clinical findings.

In the example shown in FIG. 5, a user has selected the first clinical finding (“LV is mildly dilated”), as shown by the first clinical finding being highlighted. The first clinical finding includes a link to additional information, herein shown as a plus sign, although the link may be embedded within the visualization of the clinical finding (e.g., within the textual representation of the clinical finding) or presented in another form. The link may appear only once the clinical finding is selected, or the link may be present regardless of whether the clinical finding is selected.

Due to the selection of the first clinical finding, patient GUI in the second configuration 500 may include a representative image 508, a plurality of thumbnail images 510, and a list of determinative clinical parameters. The representative image 508 may be selected due the image including one or more anatomical and/or physiological features related to the selected clinical finding. For example, the first clinical finding is directed to the left ventricle of the heart, and thus the representative image 508 includes a view of the left ventricle. The plurality of thumbnail images 510 may also be related to the selected clinical finding. For example, each thumbnail image displayed in the patient GUI may include at least part of the left ventricle and/or include anatomical and/or physiological features on which the clinical finding is based. Further, a thumbnail of the representative image is also displayed, and is shown in FIG. 5 in highlight, thereby notifying the user that the image is currently being displayed. However, the above examples are non-limiting, and other configurations are possible. For example, an image may only be displayed in response to a selection of a clinical parameter, as explained in more detail below. In another example, the representative image may be a first image acquired in the exam, a standard image (e.g., an apical four chamber view of the heart), or some other image from the exam that may or may not be related to a selected clinical finding (e.g., the image may be selected at random).

As shown herein, the listing of determinative clinical parameters 512 is displayed over the representative image, but other configurations are possible (e.g., the determinative parameters displayed alongside, but not over, the representative image). By selecting a determinative clinical parameter, an annotated image from which that determinative clinical was measured or determined may be displayed, as shown in FIG. 6 and described below. Further, the determinative clinical parameter may include a link to a trend line or graph for that determinative clinical parameter, also as shown in FIG. 6 and described below.

The listing of determinative clinical parameters 512 includes the determinative clinical parameters for the selected clinical finding, herein the left ventricle being mildly dilated. The determinative clinical parameters include left ventricle (LV) diameter during diastole (LV Diam D), LV diameter during diastole normalized by body surface area (VL Diam D/BSA), LV volume during diastole (LV Volume D), LV volume during systole (LV Volume S), and LV mass.

Returning to FIG. 3A, method 300 includes, at 322, adjusting the representative image when a different thumbnail is selected. For example, the user may select a different thumbnail image (e.g., by clicking on or entering a touch input on the thumbnail image), causing the current representative image to be replaced with the image corresponding to the newly-selected thumbnail image.

At 324, method 300 includes determining if an input selecting a link from a clinical parameter has been received. As explained above and shown in FIG. 5, one or more of the displayed determinative clinical parameters may include a link to additional information. The user may select the link with a suitable input (e.g., mouse, voice, or touch). If an input selecting a link within a clinical parameter is not received, method 300 proceeds to 328, which will be explained below. If an input selecting a link within a clinical parameter is received, method 300 proceeds to 326 to display a trend of that clinical parameter in the patient GUI and/or update the representative image, such as with an annotated image that includes a visualization of the clinical parameter relative to the image.

FIG. 6 shows the patient GUI in a third configuration 600. The patient GUI in the third configuration 600 may be displayed in response to a user selecting a link within a clinical finding and in response to the user selecting a link within a determinative clinical parameter. The patient GUI in the third configuration includes similar information as the patient GUI in the second configuration (e.g., the patient information 502, listing of clinical findings 504, user interface control buttons 506, representative image 508, thumbnail images 510, and determinative clinical parameters 512), and further includes a trend line 602 and annotation 604 of a selected clinical parameter.

As shown, LV Diam DB SA has been selected, resulting in display of a representative image 508 that includes the left ventricle, and further includes an annotation 604 of the measurement of the LV diameter. The annotation is a line having endpoints at the boundaries of the left ventricle at which the measurement was made. In this way, the clinician may be able to verify the accuracy of the measurement (e.g., verify the measurement was taken at the correct location).

The annotation may take different forms depending on the clinical parameter being visualized. For example, if the user selects the third clinical finding (AV is trileaflet and mildly thickened), the determinative parameters for the third clinical finding may include aortic valve shape. When the user selects the aortic valve shape, an image of the aortic valve showing the three leaflets may be displayed, and the annotation may include a line or other highlighting mechanism around the aortic valve. Further, confirmation of the trileaflet valve may be made by analyzing the aortic valve in both the open and closed positions. Thus, in some examples, selection of the aortic valve shape clinical parameter may result in display of more than one annotated image, e.g., an annotated image with the valve open and an annotated image with the valve closed. In another example, a clinical finding of aortic regurgitation may be visualized by displaying Doppler spectrums with measurements of backflow/velocities through the aortic valve. Also, color Doppler images may be displayed with annotations to point the attention to areas in the images with turbulent and/or backflow through the aortic valve. For ejection fraction, traces/segmentation of the LV cavity in end diastolic and end systolic frames may be displayed (also four chamber and two chamber view if made in 2D), and the user may be able to change segmentation and/or change frames. Other annotations or visualizations that draw the user's attention to how a clinical parameter and subsequent clinical finding were determined are within the scope of this disclosure

Further, at least some of the determinative clinical parameters displayed in the patient GUI may include a link to a trend line. As shown, the LV Diam DB SA clinical parameter includes a link to a trend line, which has been selected by the user. As a result, a trend line 604 is shown for the clinical parameter, herein showing the LV Diam DB SA for the patient over time.

Returning to FIG. 3B, at 328, method 300 includes determining if an input accepting a selected clinical finding has been received. The patient GUI may include a first user interface control button, in the form of an “accept” button, and a second user interface control button, in the form of a “cancel” button, as shown in FIGS. 5 and 6. If a clinician agrees with the clinical finding identified by the AI-based diagnostic assistant, the clinician may enter an input selecting the accept button, thus accepting the clinical finding. If the clinician does not agree with the clinical finding, or does not want that clinical finding included in a final report, the clinician may enter an input selecting the cancel button, thereby not accepting the clinical finding.

If an input accepting a selected clinical finding is received, method 300 proceeds to 330 to save the clinical finding in a final report. The final report may include all accepted clinical findings, and may further include the images (e.g., annotated images) and determinative clinical parameters used to generate each accepted clinical finding. If an input accepting the clinical finding is not received, method 300 proceeds to 336 to determine if an input cancelling the clinical finding has been received. If no input cancelling the clinical finding has been received, method 300 loops back to 306, 314, or 326, depending on progression through the method. In this way, the currently-displayed configuration of the patient GUI may be maintained until the user makes another selection or pauses or cancels review of the exam. If an input is received cancelling the clinical finding, method 300 proceeds to 338 to omit the clinical finding from the final report. However, in some examples, cancelling a given clinical finding may cause a modification or additional information GUI to be displayed, whereby the user may provide information explaining why the clinical finding was cancelled (e.g., insufficient data, poor image quality, etc.). Additionally or alternatively, if the user (e.g., clinician) disagrees with one or more of the clinical parameters used to identify the clinical finding, the user may modify the clinical parameter, and/or modify the clinical finding. For example, if the user disagrees with the location where the LV Diam D was measured, the user may update the measurement by moving the line showing where the diameter was measured. In response, the AI-based parameter module may update that clinical parameter, which may or may not cause a change in the identified clinical finding.

Both 330 and 338 proceed to 332, where method 300 includes determining if all clinical findings identified by the AI-based diagnostic module have been cancelled or accepted. If no, method 300 returns to 306 to output the patient GUI in the first configuration, allowing the clinician to select another clinical finding for review. If all clinical findings have been cancelled or accepted, method 300 proceeds to save the final report and/or send the final report to the patient's medical record (e.g., electronic medical record), and then method 300 ends.

A technical effect of automatically generating a set of clinical parameters from medical images of a patient and automatically identifying a clinical finding based on the set of clinical parameters is consistent measurement and determination of the clinical parameters, across all examined patients, resulting in more consistent diagnoses of clinical findings. Another technical effect is reduced exam duration by automating the measurement aspect of the exam, which may expedite diagnosis and subsequent treatment, reduce patient anxiety, and reduce clinician workload.

An example provides a method including obtaining a set of medical images of a patient; automatically generating a set of clinical parameters from the set of medical images; automatically identifying a clinical finding of the patient based on at least one selected clinical parameter of the set of clinical parameters; outputting a graphical user interface for display on a display device, the graphical user interface including a visualization of the clinical finding and a link within the visualization of the clinical finding; and responsive to selection of the link, outputting, for display within the graphical user interface, a visualization of the at least one selected clinical parameter. In a first example of the method, the link is a first link, the visualization of the at least one selected clinical parameter includes a second link, and the method further includes, responsive to selection of the second link, outputting, for display within the graphical user interface, a selected image from the set of medical images, the selected image including a visualization of the at least one selected clinical parameter in relation to the selected image. In a second example of the method, which optionally includes the first example, the method further includes outputting, for display within the graphical user interface, a set of thumbnail images each representing a respective image of the set of medical images associated with the clinical finding. In a third example of the method, which optionally includes one or both of the first and second examples, the set of clinical parameters comprises one or more of: one or more quantitative measurements of anatomical and/or physiological features of the patient, one or more classifications of anatomical and/or physiological features of the patient, and one or more scores of anatomical and/or physiological features of the patient; and automatically identifying the clinical finding of the patient comprises automatically identifying the clinical finding based on at least one selected clinical parameter of the set of clinical parameters and patient information received from one or more of a remote computing device and user input. In a fourth example of the method, which optionally includes one or more or each of the first through third examples, the one or more quantitative measurements include one or more of: one or more distance measurements, one or more area measurements, one or more volume measurements, one or more velocity measurements, and one or more time measurements. In a fifth example of the method, which optionally includes one or more or each of the first through fourth examples, automatically generating the set of clinical parameters from the set of medical images comprises automatically identifying each anatomical feature and each physiological feature in each image of the set of medical images and generating the one or more quantitative measurements by measuring at least one identified anatomical feature and/or identified physiological feature. In a sixth example of the method, which optionally includes one or more or each of the first through fifth examples, obtaining the set of medical images of the patient comprises obtaining a set of ultrasound images of the patient. In a seventh example of the method, which optionally includes one or more or each of the first through sixth examples, obtaining the set of ultrasound images comprises obtaining a set of ultrasound images of a heart of the patient, and wherein automatically generating the set of clinical parameters from the set of medical images comprises automatically generating the set of clinical parameters from the set of ultrasound images of the heart according to an echocardiogram protocol. In an eighth example of the method, which optionally includes one or more or each of the first through seventh examples, obtaining the set of medical images of the patient comprises obtaining a set of images of a heart of the patient, the set of images of the heart of the patient acquired from an ultrasound imaging system, a magnetic resonance imaging system, or a computed tomography imaging system. In a ninth example of the method, which optionally includes one or more or each of the first through eighth examples, automatically identifying the clinical finding of the patient comprises automatically identifying the clinical finding based on at least one selected clinical parameter of the set of clinical parameters and further based on prior clinical findings of the patient identified from a previous exam. In a tenth example of the method, which optionally includes one or more or each of the first through ninth examples, the method further includes identifying one or more clinical findings for each of a plurality of patients; assigning a severity score to each patient based on the identified one or more clinical findings; and outputting a second graphical user interface that presents a list of the plurality of patients sorted by respective severity scores.

An example provides a workstation, comprising a display and a processor configured to execute instructions stored in memory to: output, to the display, a visualization of a clinical finding of a patient, the clinical finding automatically identified from a clinical parameter of a set of clinical parameters automatically determined from a set of medical images of the patient, the visualization of the clinical finding including a link; and responsive to a user selecting the link, output to the display an image from the set of medical images, the image associated with the clinical finding and including a visualization of the clinical parameter. In a first example of the workstation, the processor is configured to execute instructions to output, to the display, a visualization of the clinical parameter along with the visualization of the clinical finding. In a second example of the workstation, which optionally includes the first example, the processor is configured to execute instructions to output, to the display, a visualization of the clinical parameter along with the image. In a third example of the workstation, which optionally includes one or both of the first and second examples, the clinical parameter includes a quantitative measurement of an anatomical feature of the patient, wherein the clinical finding is identified based on the quantitative measurement having a value outside a normal range of values for the quantitative measurement, and wherein the image includes the anatomical feature annotated with a visualization of the quantitative measurement. In fourth example of the workstation, which optionally includes one or more or each of the first through third examples, the processor is configured to execute instructions to: output, to the display, a first user interface control button and a second user interface control button; responsive to the user selecting the first user interface control button, save the clinical finding in a report; and responsive to the user selecting the second user interface control button, omit the clinical finding from the report, the report configured to be saved in a medical record of the patient.

Another example provides a workstation, comprising a display and a processor configured to execute instructions stored in memory to: output, to the display, a graphical user interface including a visualization of a clinical finding of a patient and a visualization of one or more clinical parameters, the clinical finding automatically identified from the one or more clinical parameters, the one or more clinical parameters automatically generated from a set of medical images of the patient; and responsive to a user selecting a link associated with a selected clinical parameter of the one or more clinical parameters, output, to the display, an image from the set of medical images that includes an annotation of the selected clinical parameter. In a first example of the workstation, the link is a first link associated with a first selected clinical parameter, the image is a first image, and the processor is configured to execute instructions to, responsive to the user selecting a second link associated with a second selected clinical parameter of the one or more clinical parameters, output, to the display, a second image from the set of medical images that includes an annotation of the second selected clinical parameter. In a second example of the workstation, which optionally includes the first example, the one or more clinical parameters comprise a subset of a plurality of clinical parameters automatically generated from the set of medical images of the patient, and the clinical finding is a first clinical finding of a plurality of clinical findings automatically identified from the plurality of clinical parameters. In a third example of the workstation, which optionally includes one or both of the first and second examples, the first clinical finding comprises an abnormal finding, at least one other clinical finding of the plurality of clinical findings comprises a normal finding, the one or more clinical parameters includes a first quantitative measurement of an anatomical feature of the patient, the first clinical finding is identified based on the first quantitative measurement having a value outside a normal range of values for the first quantitative measurement, the image includes the anatomical feature annotated with a visualization of the first quantitative measurement, and the normal finding is identified based on a second quantitative measurement of an anatomical feature of the patient having a value within a normal range of values for the second quantitative measurement.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
obtaining a set of medical images of a patient;
automatically generating a set of clinical parameters from the set of medical images;
automatically identifying a clinical finding of the patient based on at least one selected clinical parameter of the set of clinical parameters;
outputting a graphical user interface for display on a display device, the graphical user interface including a visualization of the clinical finding and a link within the visualization of the clinical finding; and
responsive to selection of the link, outputting, for display within the graphical user interface, a visualization of the at least one selected clinical parameter.

2. The method of claim 1, wherein the link is a first link, the visualization of the at least one selected clinical parameter includes a second link, and further comprising responsive to selection of the second link, outputting, for display within the graphical user interface, a selected image from the set of medical images, the selected image including a visualization of the at least one selected clinical parameter in relation to the selected image.

3. The method of claim 2, further comprising outputting, for display within the graphical user interface, a set of thumbnail images each representing a respective image of the set of medical images associated with the clinical finding.

4. The method of claim 1, wherein:
the set of clinical parameters comprises one or more of: one or more quantitative measurements of anatomical and/or physiological features of the patient, one or more classifications of anatomical and/or physiological features of the patient, and one or more scores of anatomical and/or physiological features of the patient; and
automatically identifying the clinical finding of the patient comprises automatically identifying the clinical finding based on at least one selected clinical parameter of the set of clinical parameters and patient information received from one or more of a remote computing device and user input.

5. The method of claim 4, wherein the one or more quantitative measurements include one or more of: one or more distance measurements, one or more area measurements, one or more volume measurements, one or more velocity measurements, and one or more time measurements.

6. The method of claim 5, wherein automatically generating the set of clinical parameters from the set of medical images comprises automatically identifying each anatomical feature and each physiological feature in each image of the set of medical images and generating the one or more quantitative measurements by measuring at least one identified anatomical feature and/or identified physiological feature.

7. The method of claim 1, wherein obtaining the set of medical images of the patient comprises obtaining a set of ultrasound images of the patient.

8. The method of claim 7, wherein obtaining the set of ultrasound images comprises obtaining a set of ultrasound images of a heart of the patient, and wherein automatically generating the set of clinical parameters from the set of medical images comprises automatically generating the set of clinical parameters from the set of ultrasound images of the heart according to an echocardiogram protocol.

9. The method of claim 1, wherein obtaining the set of medical images of the patient comprises obtaining a set of images of a heart of the patient, the set of images of the heart of the patient acquired from an ultrasound imaging system, a magnetic resonance imaging system, or a computed tomography imaging system.

10. The method of claim 9, wherein automatically identifying the clinical finding of the patient comprises automatically identifying the clinical finding based on at least one selected clinical parameter of the set of clinical parameters and further based on prior clinical findings of the patient identified from a previous exam.

11. The method of claim 1, further comprising:
identifying one or more clinical findings for each of a plurality of patients;
assigning a severity score to each patient based on the identified one or more clinical findings; and
outputting a second graphical user interface that presents a list of the plurality of patients sorted by respective severity scores.

12. A workstation, comprising:
a display; and
a processor configured to execute instructions stored in memory to:
output, to the display, a visualization of a clinical finding of a patient, the clinical finding automatically identified from a clinical parameter of a set of clinical parameters automatically determined from a set of medical images of the patient, the visualization of the clinical finding including a link; and
responsive to a user selecting the link, output to the display an image from the set of medical images, the image associated with the clinical finding and including a visualization of the clinical parameter.

13. The workstation of claim 12, wherein the processor is configured to execute instructions to output, to the display, a visualization of the clinical parameter along with the visualization of the clinical finding.

14. The workstation of claim 12, wherein the processor is configured to execute instructions to output, to the display, a visualization of the clinical parameter along with the image.

15. The workstation of claim 12, wherein the clinical parameter includes a quantitative measurement of an anatomical feature of the patient, wherein the clinical finding is identified based on the quantitative measurement having a value outside a normal range of values for the quantitative measurement, and wherein the image includes the anatomical feature annotated with a visualization of the quantitative measurement.

16. The workstation of claim 12, wherein the processor is configured to execute instructions to:
output, to the display, a first user interface control button and a second user interface control button;
responsive to the user selecting the first user interface control button, save the clinical finding in a report; and
responsive to the user selecting the second user interface control button, omit the clinical finding from the report, the report configured to be saved in a medical record of the patient.

17. A workstation, comprising:
a display; and
a processor configured to execute instructions stored in memory to:
output, to the display, a graphical user interface including a visualization of a clinical finding of a patient and a visualization of one or more clinical parameters, the clinical finding automatically identified from the one or more clinical parameters, the one or more clinical parameters automatically generated from a set of medical images of the patient; and
responsive to a user selecting a link associated with a selected clinical parameter of the one or more clinical parameters, output, to the display, an image from the set of medical images that includes an annotation of the selected clinical parameter.

18. The workstation of claim 17, wherein the link is a first link associated with a first selected clinical parameter, wherein the image is a first image, and wherein the processor is configured to execute instructions to, responsive to the user selecting a second link associated with a second selected clinical parameter of the one or more clinical parameters, output, to the display, a second image from the set of medical images that includes an annotation of the second selected clinical parameter.

19. The workstation of claim 17, wherein the one or more clinical parameters comprise a subset of a plurality of clinical parameters automatically generated from the set of medical images of the patient, and wherein the clinical finding is a first clinical finding of a plurality of clinical findings automatically identified from the plurality of clinical parameters.

20. The workstation of claim 19, wherein the first clinical finding comprises an abnormal finding, wherein at least one other clinical finding of the plurality of clinical findings comprises a normal finding, wherein the one or more clinical parameters includes a first quantitative measurement of an anatomical feature of the patient, wherein the first clinical finding is identified based on the first quantitative measurement having a value outside a normal range of values for the first quantitative measurement, wherein the image includes the anatomical feature annotated with a visualization of the first quantitative measurement, and wherein the normal finding is identified based on a second quantitative measurement of an anatomical feature of the patient having a value within a normal range of values for the second quantitative measurement.
